(19) United States
(12) Patent Application Publication
(10) Pub. No.: US 2025/0143538 A1
(43) Pub. Date: May 8, 2025

(54) IMAGE PROCESSING DEVICE, IMAGE PROCESSING METHOD, AND STORAGE MEDIUM

(71) Applicant: OLYMPUS MEDICAL SYSTEMS CORP., Tokyo (JP)

(72) Inventors: Yoshioki KANEKO, Tokyo (JP); Shunsuke OKUMURA, Tokyo (JP)

(73) Assignee: OLYMPUS MEDICAL SYSTEMS CORP., Tokyo (JP)

(21) Appl. No.: 18/937,466

(22) Filed: Nov. 5, 2024

Related U.S. Application Data
(60) Provisional application No. 63/547,594, filed on Nov. 7, 2023.

(51) Int. Cl. A61B 1/00; A61B 1/015; A61B 1/06; A61B 18/14; G06T 17/00

(52) U.S. Cl. CPC A61B 1/00006; A61B 1/00009; A61B 1/015; A61B 1/0655; G06T 17/00; A61B 18/1492; G06T 2210/41

(57) ABSTRACT

An image processing device for used with an endoscope system. One or more processors of the image processing device are configured to create a three-dimensional model of a subject from a group of images captured by an endoscope in the endoscope system, detect a non-observed region in which an image from the group of images is not captured by the endoscope based on the three-dimensional model, acquire operation signals of the endoscope system, and determine whether the operation signal matches a condition, responsive to determining the operation signal matches the condition, temporarily stop detecting the non-observed region. The condition includes an operation of the endoscope system that causes the group of images has a missing portion.

### CROSS-REFERENCE TO RELATED APPLICATIONS

This application claims the benefit of U.S. Provisional Application No. 63/547,594, filed Nov. 7, 2023, which is hereby incorporated by reference herein in its entirety.

### TECHNICAL FIELD

The present disclosure relates to an image processing device, an image processing method, and a storage medium.

### BACKGROUND ART

Conventionally, there is a known technology for creating a three-dimensional model of an imaging subject from an image group acquired by means of an endoscope (for example, see PTL 1). According to PTL 1, a blank region for which a three-dimensional model is not created is determined to be a non-observed region that is not observed by means of the endoscope, and the non-observed region is displayed in a visually recognizable manner.

### SUMMARY

An aspect of the present disclosure is an image processing device for use with an endoscope system, the image processing device comprising: one or more processors comprising hardware, wherein the one or more processors being configured to: create a three-dimensional model of a subject from a group of images captured by an endoscope in the endoscope system; detect a non-observed region in which an image from the group of images is not captured by the endoscope based on the three-dimensional model; acquire operation signals of the endoscope system; and determine whether the operation signal matches a condition, wherein the condition includes an operation of the endoscope system that causes the group of images has a missing portion; responsive to determining the operation signal matches the condition, temporarily stop detecting the non-observed region.

An aspect of the present disclosure is an image processing method comprising: creating a three-dimensional model of a subject from a group of images captured by an endoscope; detecting a non-observed region in which an image from the group of images is not captured by the endoscope based on the three-dimensional model; acquiring operation signals of an endoscope system including the endoscope; determining whether the operation signal matches a condition, wherein the condition includes an operation of the endoscope system that causes the group of images has a missing portion; responsive to determining the operation signal matches the condition, temporarily stopping detection of the non-observed region.

An aspect of the present disclosure is a computer-readable, non-transitory storage medium configured to cause a computer to execute: creation of a three-dimensional model of a subject from a group of images captured by an endoscope; detection of a non-observed region in which an image from the group of images is not captured by the endoscope based on the three-dimensional model; acquisition of operation signals of an endoscope system including the endoscope; and determination of whether the operation signal matches a condition, wherein the condition includes an operation of the endoscope system that causes the group of images has a missing portion; responsive to determining the operation signal matches the condition, temporary stopping the detection of the non-observed region.

### BRIEF DESCRIPTION OF DRAWINGS

FIG. 1 is a block diagram showing overall configurations of an image processing device and an endoscope system according to a first embodiment.

FIG. 2 is a diagram for explaining colonoscopy.

FIG. 3 is a diagram for explaining three-dimensional model creation in the colonoscopy.

FIG. 4 is a diagram for explaining an image displayed on a display device in the colonoscopy.

FIG. 5 is a flowchart of an image processing method according to the first embodiment.

FIG. 6 is a flowchart of a modified example of the image processing method of the first embodiment.

FIG. 7 is a diagram showing a selection screen for a user to select certain conditions.

FIG. 8A is a diagram showing an example of a post-colonoscopy report.

FIG. 8B is a diagram showing another example of the post-colonoscopy report.

FIG. 9 is a block diagram showing overall configurations of an image processing device and an endoscope system according to a second embodiment.

FIG. 10 is a diagram showing the correspondence relationship between combinations of endoscope system devices and the certain conditions.

FIG. 11 is a flowchart of a modified example of an image processing method of the second embodiment.

### DESCRIPTION OF EMBODIMENTS

### First Embodiment

Conventionally, there are cases in which an image group is missing a portion thereof due to the operations of an endoscope system. A three-dimensional model created from the image group that is missing the portion thereof lacks accuracy, and, consequently, a problem could occur in the determination related to the non-observed region.

An image processing device, an image processing method, an image processing program, and a storage medium according to a first embodiment of the present disclosure will be described with reference to the drawings.

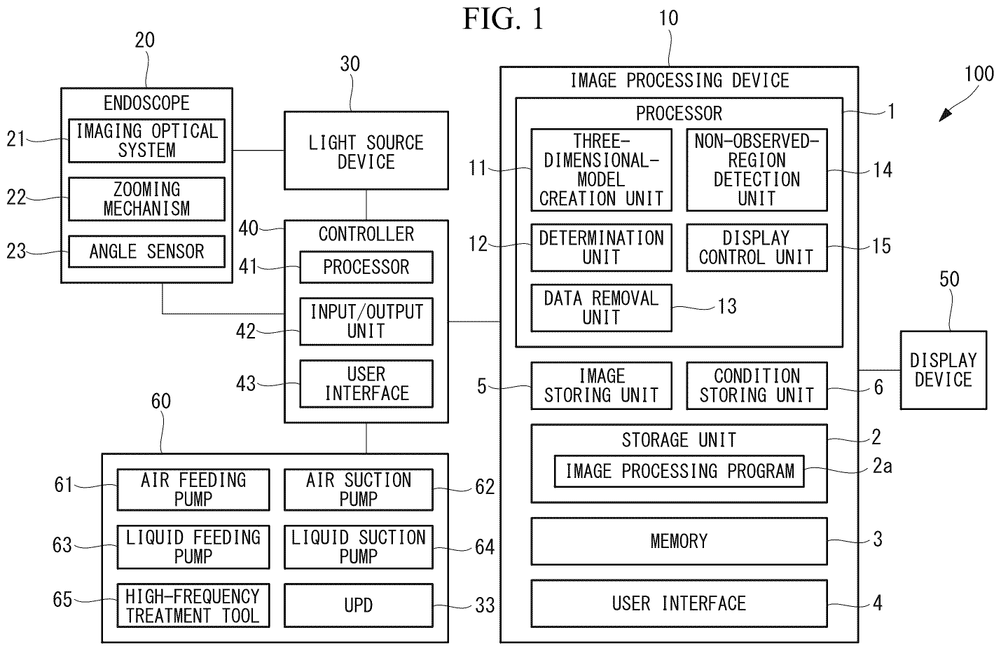

As shown in FIG. 1, an image processing device 10 according to this embodiment is applied to an endoscope system 100.

The endoscope system 100 includes the image processing device 10, an endoscope 20, a light source device 30, a controller 40, and a display device 50. In addition, the endoscope system 100 includes peripheral devices 60 of the endoscope 20 used in endoscopy. The peripheral devices 60 include, for example, an air feeding pump 61, an air suction pump 62, a liquid feeding pump 63, a liquid suction pump 64, a high-frequency treatment tool 65, and an endoscope-insertion-shape observation device (UPD) 66.

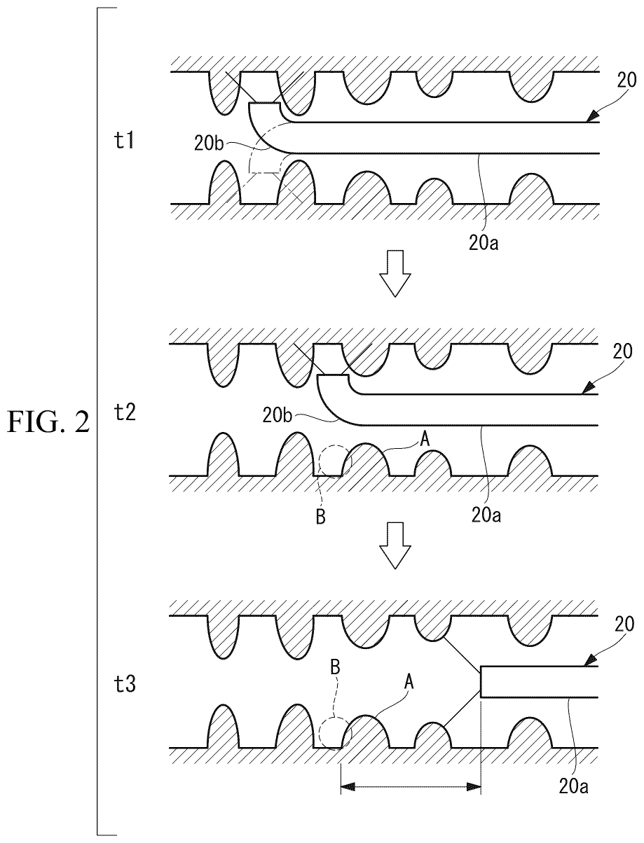

The endoscope 20 is, for example, a flexible endoscope for a digestive organ, such as the colon. The endoscope 20 has an elongated, flexible insertion portion 20a, a bending portion 20b provided at a distal-end portion of the insertion portion 20a, and an operation unit (not shown) connected to a basal end of the insertion portion 20a (see FIG. 2).

In addition, the endoscope 20 has an imaging optical system 21 that captures an image of an imaging subject, a zooming mechanism 22 for enlarging and shrinking the imaging subject in the image, and an angle sensor 23 that detects the bending angle of the bending portion 20b. The imaging optical system 21 includes an objective lens and an imaging element, such as a CMOS image sensor. The zooming mechanism 22 changes the magnification for the imaging subject in the image by means of optical zooming or digital zooming. For example, the zooming mechanism 22 switches the magnification from a normal magnification to a high magnification as a result of a user tuning on a zoom switch provided in the operation unit.

The light source device 30 is connected to the endoscope 20 and provides the endoscope 20 with illumination light. The light source device 30 has LEDs of multiple colors and is capable of outputting multiple types of illumination light as a result of the respective LEDs being turned ON/OFF.

For example, the light source device 30 has five types of LEDs in colors of violet, blue, green, amber, and red. The multiple types of illumination light include white light for normal observation and special light for special-light observation. The white light is formed from the light of the five colors. The special light is formed from, for example, the blue and green light for narrow band imaging (NBI).

The controller 40 includes a processor 41, an input/output unit 42, and a user interface 43. The input/output unit 42 has a known input/output interface, and the controller 40 is connected to the image processing device 10, the endoscope 20, the light source device 30, and the peripheral devices 60 via the input/output unit 42. The image captured by the endoscope 20 is input to the display device 50 via the controller 40 and the image processing device 10 and is displayed on the display device 50. The display device 50 is an arbitrary type of display, such as a liquid crystal display.

The controller 40 controls the operations of the light source device 30 and the peripheral devices 60. For example, the processor 41 generates, on the basis of the user operations of the user interface 43, control signals for controlling the light source device 30 and the peripheral devices 60 and transmits the control signals to the light source device 30 and the peripheral devices 60.

The image processing device 10 includes a processor 1, such as a central processing unit, a storage unit 2, a memory 3, and a user interface 4. For example, the image processing device 10 consists of an arbitrary computer, such as a personal computer.

The storage unit 2 is a computer-readable, non-transitory storage medium and is, for example, a known magnetic disk, optical disk, flash memory, or the like. The storage unit 2 stores an image processing program 2a that causes the processor 1 to execute the image processing method, described later.

The memory 3 consists of a volatile storage device, such as a random-access memory (RAM), and is used as a work area for the processor 1.

The user interface 4 has an input device, such as a mouse, a keyboard, or a touchscreen, and receives the user operations of the input device.

The processor 1 includes, as functional units, a three-dimensional (3D) model creation unit 11, a determination unit 12, a data removal unit 13, a non-observed-region detection unit 14, and a display control unit 15.

In addition, the image processing device 10 includes an image storing unit 5 that stores an image group to be used in three-dimensional model creation and a condition storing unit 6 that stores certain conditions for temporarily stopping the non-observed region detection. The storing units 5, 6 consists of, for example, the storage unit 2, the memory 3, or another storage device.

The processor 1 stores, in the image storing unit 5, the images input to the image processing device 10 from the endoscope 20 via the controller 40 and generates an image group consisting of the plurality of images for the three-dimensional model creation. The processor 1 may store all of the images input to the image processing device 10 or may select images that are suitable for the three-dimensional model creation and store said images.

The three-dimensional-model creation unit 11 acquires the image group from the image storing unit 5 and creates a three-dimensional model from the image group, the three-dimensional model representing the 3D shape of the imaging subject in said image group. A known three-dimensional reconstruction technology, such as visual Simultaneous Localization and Mapping (SLAM), is used in the three-dimensional model creation.

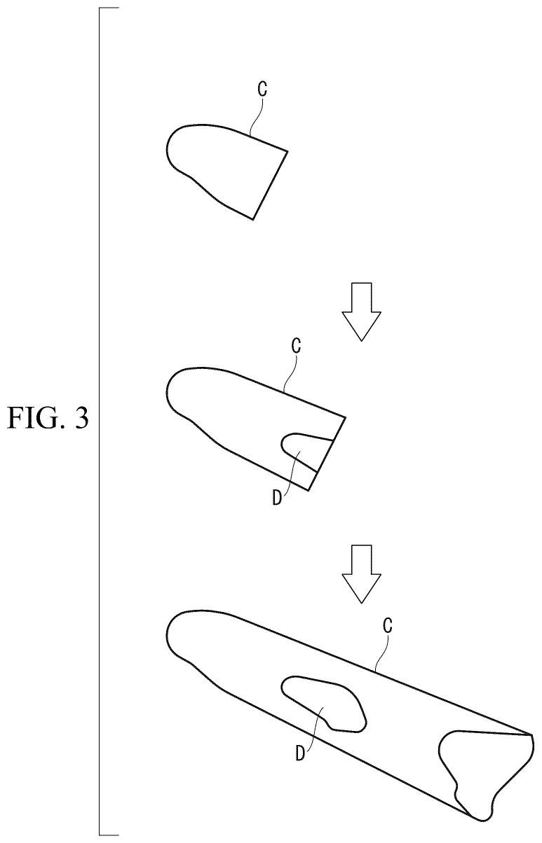

FIGS. 2 and 3 respectively describe colonoscopy and colon three-dimensional model creation.

In colonoscopy, after the endoscope 20 is inserted from the anus so as to reach the cecum, the cecum, the ascending colon, the transverse colon, the descending colon, the sigmoid colon, and the rectum are observed in order while the endoscope 20 is retracted toward the anus. In order to observe a mucous membrane of the colon, the mucous membrane is cleaned by performing feeding a liquid thereto, feeding air, and sucking the air, as needed. In the case in which a lesion is found, a magnified observation or a special light observation of the lesion is performed and treatment using the high-frequency treatment tool 65 is performed, as needed.

As shown in FIG. 3, a three-dimensional model C is created in order from the cecum as the endoscope 20 is moved. The three-dimensional model C could include a missing portion D in which an imaging subject model is not created. The missing portion D corresponds to a non-observed region B in which the imaging subject is not observed by means of the endoscope 20. For example, in colonoscopy, an area in a back side of a fold A, which tends to be in a blind spot, could be the non-observed region B. In FIGS. 2 and 3, a non-observed region B occurs on the back side of the fold A at a time t2, and a missing portion D corresponding to the non-observed region B is formed in the three-dimensional model C.

In order to create a three-dimensional model C that is continuous over the movement range of the viewing field of the endoscope 20, the imaging subject needs to be continuous in the plurality of images used in creating the three-dimensional model C. However, due to the operations of the endoscope system 100, there are cases in which images that do not have information about the shape of the imaging subject are input to the image processing device 10 or images are temporarily not input to the image processing device 10. In such a case, a portion of the image group becomes missing and, for example, some of the images in the image group do not have the information about the shape of the imaging subject or the image group does not contain images of some regions of the imaging subject. Therefore, the image group that is missing a portion of the imaging subject contains a portion in which the imaging subject is discontinuous and that does not have the information about the shape of the imaging subject.

For example, when the illumination light is switched to the NBI special light from the white light, the intensity of the illumination light temporarily drops and the endoscope 20 temporarily acquires dark images that do not have the information about the shape of the imaging subject. In an image group containing the dark images, a portion of the imaging subject corresponding to the dark images becomes a discontinuous portion.

The three-dimensional-model creation unit 11 continues to create the three-dimensional model C regardless of whether or not the image group is missing a portion of the imaging subject. The three-dimensional model C created from the image group that is missing the portion contains a missing portion corresponding to the missing portion of the image group.

The determination unit 12 acquires operation signals of the endoscope system 100 and compares the operation signals with the certain conditions stored in the condition storing unit 6. The determination unit 12 determines whether or not the operation signals match the certain conditions.

The operation signals indicate the operations of devices 20, 30, and 61 to 66 constituting the endoscope system 100 and contain, for example, signals that indicate predetermined operations of the devices 20, 30, and 61 to 66 that generate the missing portion of the image group. The determination unit 12 acquires the operation signals from the controller 40 or the devices 20, 30, and 61 to 66.

Examples of the operation signals include signals that indicate switching of the types of the illumination light provided to the endoscope 20 from the light source device 30, and are, for example, control signals that the controller 40 outputs to the light source device 30 in order to cause the said device to execute the switching of the types of the illumination light. The determination unit 12 acquires the control signals from the controller 40.

The certain conditions are the predetermined operations of the endoscope system 100 that generate the missing portion in the image group. For example, the certain conditions include switching of the types of the illumination light provided to the endoscope 20 from the light source device 30.

The data removal unit 13 detects, in the case in which the operation signals match the certain conditions, the missing portion in the three-dimensional model C, which is the portion corresponding to the certain conditions (in other words, the portion corresponding to the missing portion of the image group) and removes the missing portion data from the three-dimensional model C. Accordingly, the three-dimensional model C that does not contain the missing portion due to the operations of the endoscope system 100 is created.

The data removal unit 13 may be used to generate the 3D model C by selecting another image for generating the 3D model C instead of removing or in addition to removing the data of the missing portion. Among the image groups, select images that are adjacent to the missing part of the image group chronologically and use them to generate the 3D model. The image that is chronologically adjacent to the missing portion of the image group is not limited to the next image chronologically and may be selected from among the images acquired within a predetermined time from the timing when a predetermined condition occurs. Further, when switching of the illumination light type is performed, an image acquired by the illumination light before the illumination light switching is executed may be selected instead of the missing part of the image group. In this way, the effect of stopping the detection of the unobserved region can be minimized.

The non-observed-region detection unit 14 detects, on the basis of the determination result of the determination unit 12, the non-observed region in the three-dimensional model C.

Specifically, in the case in which it is determined that the operation signals do not match the certain conditions, the non-observed-region detection unit 14 detects, as a non-observed region, the missing portion D in the three-dimensional model C created by the three-dimensional-model creation unit 11.

On the other hand, in the case in which it is determined that the operation signals match the certain conditions, the non-observed-region detection unit 14 temporarily stops the non-observed region detection. After the missing portion due to the operations of the endoscope system 100 is removed by the data removal unit 13, the non-observed-region detection unit 14 resumes the non-observed region detection and detects, as a non-observed region, the missing portion D in the three-dimensional model C from which the missing portion due to the operations of the endoscope system 100 has been removed.

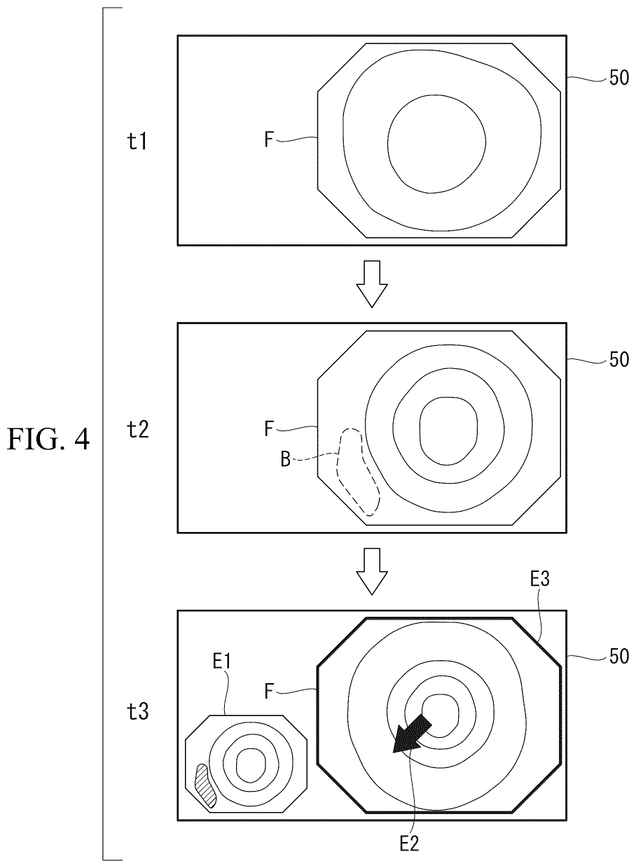

As shown in FIG. 4, the display control unit 15 generates displayed pieces E1, E2, and E3 indicating the non-observed region detection and outputs the displayed pieces E1, E2, and E3 to the display device 50 together with a current image F to be displayed on the display device 50. The display control unit 15 may output the displayed pieces E1, E2, and E3 together with an image F at a position that is away from the non-observed region D by a certain distance or an image F acquired a certain amount of time after the detection of the non-observed region D.

In the example in FIG. 4, the non-observed region D is detected at the time t2 and the displayed pieces E1, E2, and E3 are displayed on the display device 50 at a subsequent time t3.

An image E1 at the time at which the non-observed region D is detected is an example of the displayed piece, and a marker may be given to the non-observed region B in the image E1. An arrow E2 that indicates the position of the non-observed region B in the current image F is another example of the displayed piece. A frame E3 that is given to the image F and that has a predetermined color is another example of the displayed piece. As a result of at least one of the displayed pieces E1, E2, and E3 being displayed together with the image F, the presence of the non-observed region B is notified to the user.

Next, the image processing method executed by the image processing device 10 will be described.

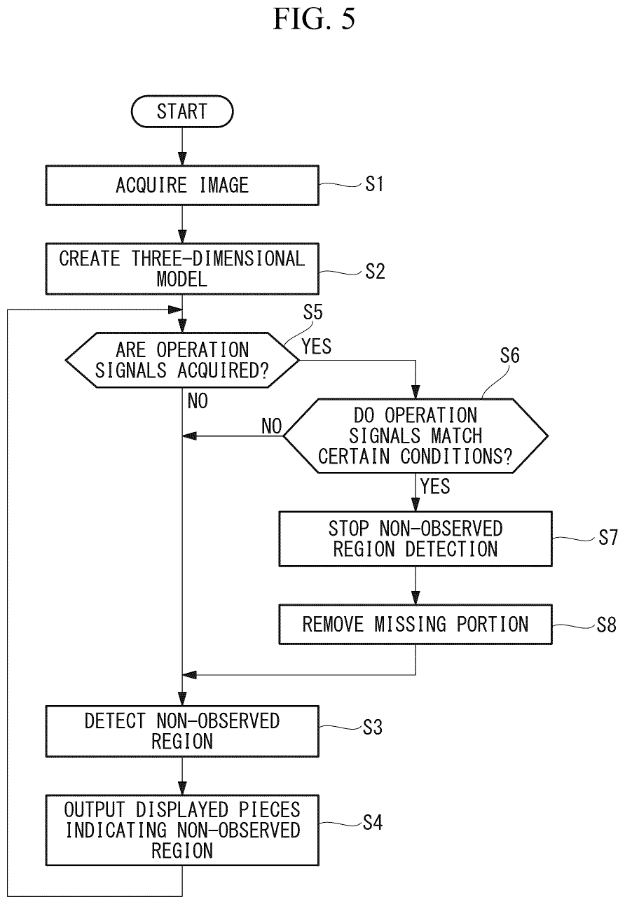

As shown in FIG. 5, the image processing method according to this embodiment includes: a step S1 of acquiring the images captured by the endoscope 20; a step S2 of creating the three-dimensional model C; a step S3 of detecting the non-observed region D in the three-dimensional model C; and a step S4 of creating and outputting the displayed pieces indicating the non-observed region D. Furthermore, the image processing method includes: a step S5 of acquiring the operation signals of the endoscope system 100; a step S6 of determining whether or not the operation signals match the certain conditions; a step S7 of temporarily stopping the non-observed region detection; and a step S8 of removing the missing portion data from the three-dimensional model C.

The processor 1 starts the acquisition of the images input to the image processing device 10 from the endoscope 20 and sequentially stores the images in the image storing unit 5 (step S1). Next, the three-dimensional-model creation unit 11 starts the creation of the three-dimensional model C of an imaging subject from the image group (step S2). The non-observed-region detection unit 14 executes the non-observed region detection in the created three-dimensional model C (step S3). In the case in which a non-observed region D is detected, the display control unit 15 generates the displayed pieces E1, E2, and E3 indicating the detected non-observed region D and outputs the displayed pieces E1, E2, and E3 to the display device 50 together with the image F (step S4)

Here, after the creation of the three-dimensional model C is started, the determination unit 12 starts the acquisition of the operation signals of the endoscope system 100 (step S5). When the operation signals are acquired in association with the operations of the endoscope system 100 (“YES” in step S5), the determination unit 12 subsequently determines whether or not the operation signals match the certain conditions (step S6).

In the case in which the operation signals do not match the certain conditions (“NO” in step S6), step S3 is executed next.

On the other hand, in the case in which the operation signals match the certain conditions (“YES” in step S6), the non-observed-region detection unit 14 temporarily stops the non-observed region detection (step S7), and the data removal unit 13 removes, from the three-dimensional model C, the missing portion data corresponding to the certain conditions (step S8). After the missing portion data are removed, the non-observed-region detection unit 14 resumes the non-observed region detection (step S3).

Accordingly, with the image processing device 10 according to this embodiment, the predetermined operations of the endoscope system 100 that generate a missing portion in the image group are detected by determining whether or not the operation signals of the endoscope system 100 match the certain conditions. After the predetermined operations are detected, the non-observed region detection is temporarily stopped, the missing portion data due to the predetermined operations are removed from the three-dimensional model, and, subsequently, the non-observed region detection is resumed. Accordingly, even in the case in which a missing portion is generated in the image group, the missing portion due to the image group missing a portion thereof is prevented from being erroneously detected as the non-observed region, and, accordingly, it is possible to prevent the occurrence of a problem in the non-observed region detection.

In this embodiment, the three-dimensional-model creation unit 11 continues to create the three-dimensional model regardless of the operations of the endoscope system 100; however, alternatively, the three-dimensional model creation may be temporarily stopped in accordance with the operations of the endoscope system 100.

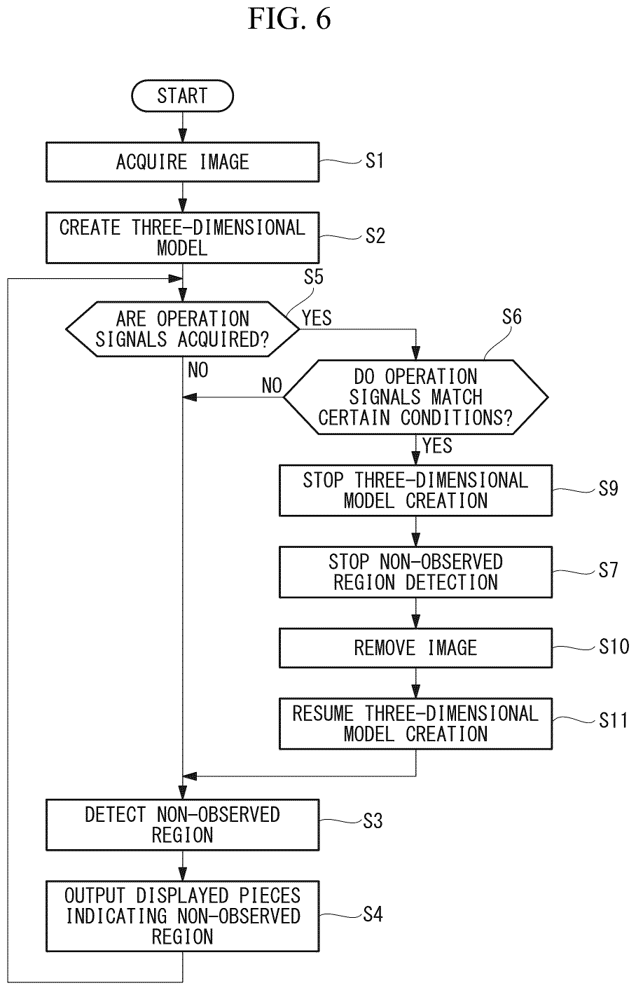

Specifically, as shown in FIG. 6, in the case in which the operation signals match the certain conditions (“YES” in step S6), the three-dimensional-model creation unit 11 temporarily stops the three-dimensional model creation (step S9) and the non-observed-region detection unit 14 temporarily stops the non-observed region detection (step S7). The data removal unit 13 removes images (matching image) corresponding to the certain conditions (for example, dark images due to switching of the illumination light) from the image group (step S10) and, subsequently, the three-dimensional-model creation unit 11 resumes the three-dimensional model creation (step S11). Therefore, the non-observed-region detection unit 14 detects, as the non-observed region, the missing portion D in the three-dimensional model C that does not contain a missing portion due to the operations of the endoscope system 100.

In the case in which the three-dimensional model creation is continued, there would be a huge amount of the three-dimensional model data, and the large amount of data needs to be stored. In addition, the processing of the data for the three-dimensional model creation is also time consuming. These problems can be eliminated by temporarily stopping the three-dimensional model creation when the predetermined operations of the endoscope system 100 are detected, and thus, it is possible to enhance the processing speed of the processor 1.

In this embodiment, in addition to when a portion of the image group is missing, the processor 1 may temporarily stop the non-observed region detection when the non-observed region detection is not necessary.

In this case, the operation signals that the determination unit 12 acquires in step S5 include signals indicating predetermined operations of the endoscope system 100 that make the non-observed region detection unnecessary. In addition, the certain conditions for the determination unit 12 to compare the operation signals in step S6 include the predetermined operations of the endoscope system 100 that make the non-observed region detection unnecessary.

The operation signals indicating the operations of the endoscope system 100 that make the non-observed region detection unnecessary include at least one of the following signals.

The first example of the operation signals is a signal that indicates a change in the magnification of the zooming mechanism 22 and is, for example, a signal for turning on the zoom switch. In this case, the certain conditions include the change in the zoom magnification of the endoscope 20 and include, for example, the switch from the normal magnification to the high magnification.

The second example of the operation signals is a signal that indicates an output of the special light from the light source device 30 and is, for example, a control signal that the controller 40 outputs to the light source device 30 in order to switch the illumination light from the white light to the special light. In this case, the certain conditions include the switching of the illumination light output by the light source device 30 to the special light.

The third example of the operation signals is a signal that indicates the activation of each of the air feeding pump 61 and the air suction pump 62 and is, for example, a control signal that the controller 40 outputs to the pump 61 or 62 to activate the pump 61 or 62. In this case, the certain conditions include the activation of the pump 61 or 62.

The fourth example of the operation signals is a signal that indicates the activation of each of the liquid feeding pump 63 and the liquid suction pump 64 and is, for example, a control signal that the controller 40 outputs to the pump 63 or 64 to activate the pump 63 or 64. In this case, the certain conditions include the activation of the pump 63 or 64.

The fifth example of the operation signals is a signal that indicates the activation of the high-frequency treatment tool 65 and is, for example, a signal for turning on the high-frequency treatment tool 65. In this case, the certain conditions include the activation of the high-frequency treatment tool 65.

The sixth example of the operation signals is a signal output by the angle sensor 23 or the UPD 66, which detects the bending angle of the bending portion 20b. In this case, the certain conditions include that the bending portion 20b being bent at a predetermined bending angle or more.

When identifying or treating a lesion, the user performs a magnified observation of the mucous membrane at the high magnification, performs a special-light observation of the lesion, treats the lesion by using the high-frequency treatment tool 65, or employs an inverted view in which a rearward observation is performed by significantly bending the bending portion 20b. While performing the identification or treatment, the non-observed region detection is unnecessary for the user.

While air is being fed and a liquid is being fed, the non-observed region detection is also unnecessary for the user.

The non-observed-region detection unit 14 may resume the non-observed region detection when a certain amount of time has passed after the non-observed region detection is temporarily stopped. For example, the non-observed-region detection unit 14 may resume the non-observed region detection when a certain amount of time has passed after the feeding of the air or the liquid is started by the activation of the pump 61 or 63.

In this embodiment, the processor 1 may set the certain conditions for temporarily stopping the non-observed region detection on the basis of the user operations of the user interface 4.

With this configuration, the user can set the conditions for temporarily stopping the non-observed region detection.

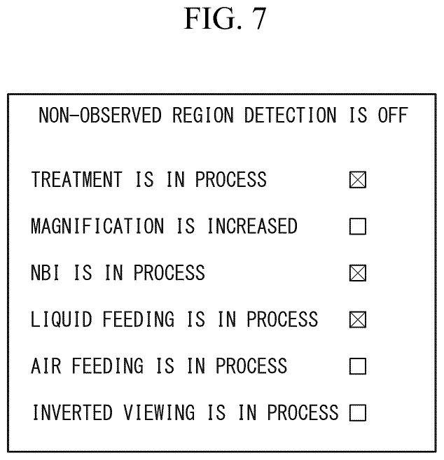

For example, as shown in FIG. 7, the processor 1 causes the display device 50 to display a selection screen containing a plurality of conditions. The user, such as a doctor, operates the user interface 4 to select desired conditions from the plurality of conditions. In FIG. 7, the conditions are selected as a result of check boxes being checked. The processor 1 sets the selected conditions to be the certain conditions and the determination unit 12 determines whether or not the operation signals match the selected conditions.

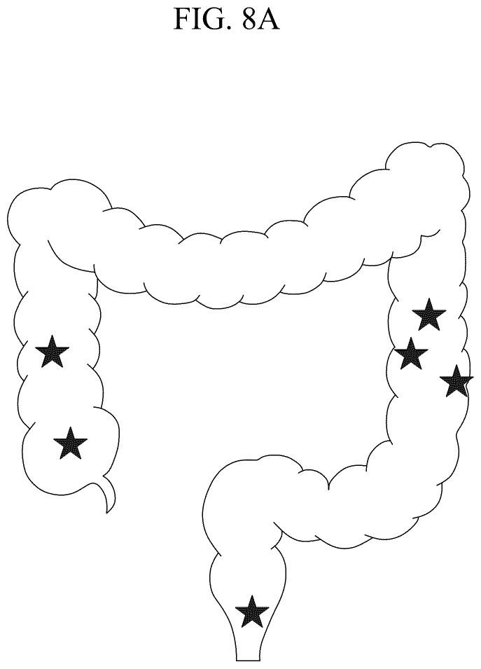

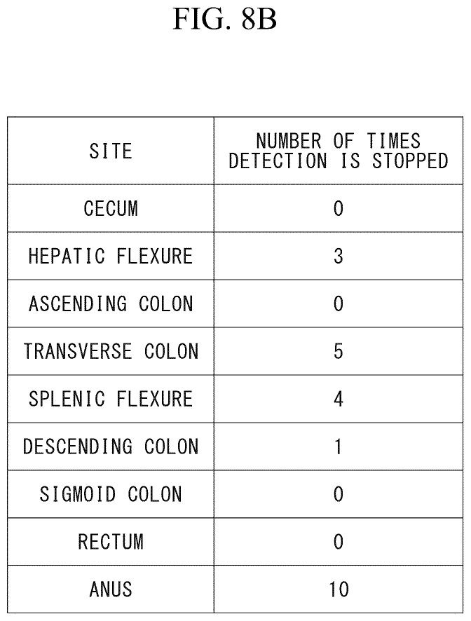

In this embodiment, the display control unit 15 may create a report related to the stopping of the non-observed region detection after the endoscopy is ended. FIGS. 8A and 8B show example reports.

The report in FIG. 8A includes a schema diagram of the colon and markers are given to positions at which the non-observed region detection was stopped. The report in FIG. 8B is a table indicating the number of times the non-observed region detection was stopped at each site of the colon.

### Second Embodiment

Next, an image processing device, an image processing method, an image processing program, and a storage medium according to a second embodiment of the present disclosure will be described.

This embodiment differs from the first embodiment in that the certain conditions are separately set for combinations of devices constituting the endoscope system.

In this embodiment, configurations that are different from the first embodiment will be described, the same reference signs are given to configurations that are the same as those of the first embodiment, and the descriptions thereof will be omitted.

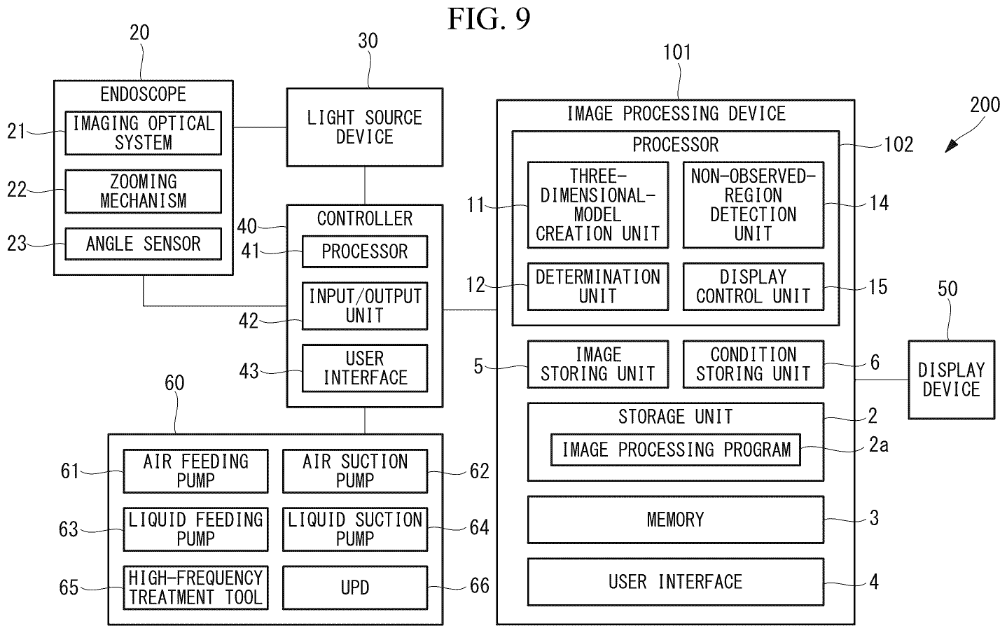

As shown in FIG. 9, an image processing device 101 according to this embodiment is applied to an endoscope system 200 including the endoscope 20, the light source device 30, the controller 40, the display device 50, and the peripheral devices 60.

The image processing device 101 has a processor 102, the storage unit 2, the memory 3, the user interface 4, the image storing unit 5, and the condition storing unit 6.

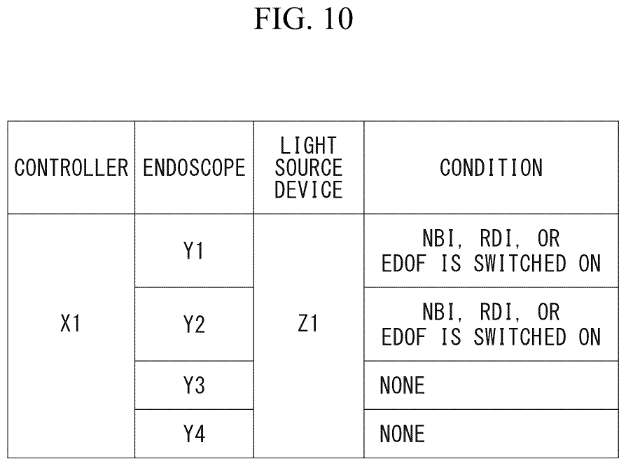

FIG. 10 shows device model combinations of the devices constituting the endoscope system 200 and the certain conditions to which the respective combinations correspond. The condition storing unit 6 stores the correspondence relationship between the device model combinations and the certain conditions.

As shown in FIG. 10, one of a plurality of device models “Y1”, “Y2”, “Y3”, and “Y4” of the endoscope 20 could be used in the endoscope system 200. The endoscope 20 has different functions depending on the device model. Similarly, one of a plurality of device models of the light source device 30 and one of a plurality of device models of the controller 40 could be used in the endoscope system 200.

In the example in FIG. 10, the controller 40 of the device model “X1” is compatible with the functions of the NBI, Red Dichromatic Imaging (RDI), and Extended Depth of Field (EDOF). The RDI is an observation method in which amber, green, and red light is radiated onto an imaging subject as the illumination light in order to facilitate viewing of blood vessels and bleeding in a deep part. The EDOF is a technology that generates an image in which a large area is in focus from two images in which a near point and a far point are in focus, respectively.

The endoscopes 20 of the device models “Y1” and “Y2” have functions of the NBI, the RDI, and the EDOF. The endoscopes 20 of the device models “Y3” and “Y4” do not have the functions of the NBI and the RDI and are capable only of the normal observation using the white light.

The light source device 30 of the device model “Z1” has LEDs of five colors, namely, violet, blue, green, amber, and red.

For example, the certain condition corresponding to the combination of “X1”, “Y1” and “Z1” is the operation for turning on the switch for executing the function of the NBI, the RDI, or the EDOF.

There is no certain condition that corresponds to the combination of “X1”, “Y3”, and “Z1”.

The processor 102 includes, as functional units, the three-dimensional-model creation unit 11, the determination unit 12, the non-observed-region detection unit 14, and the display control unit 15.

The determination unit 12 acquires, for example, system information from the controller 40. The system information is device model information of the devices 20, 30, and 40 that constitute the endoscope system 200 and that are directly or indirectly connected to the image processing device 101. The determination unit 12 acquires the certain conditions corresponding to the combination of the device models of the devices 20, 30, and 40 from the condition storing unit 6, compares the operation signals with the certain conditions, and determines whether or not the operation signals match the certain conditions.

In the case in which it is determined that the operation signals do not match the certain conditions, the non-observed-region detection unit 14 detects the missing portion D in the three-dimensional model created by the three-dimensional-model creation unit 11 as the non-observed region.

In the case in which it is determined that the operation signals match the certain conditions, the non-observed-region detection unit 14 temporarily stops the non-observed region detection.

Next, the image processing method executed by the image processing device 101 will be described.

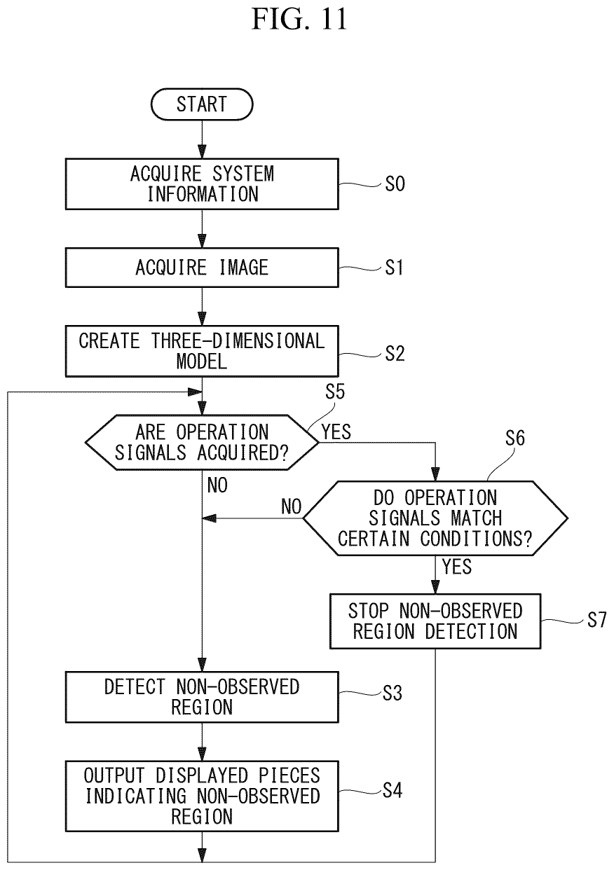

As shown in FIG. 11, the image processing method according to this embodiment includes a step S0 of acquiring the system information and steps S1 to S7.

For example, when the endoscope 20 is connected to the controller 40, the controller 40 inputs the system information to the image processing device 101. The processor 102 acquires the system information input to the image processing device 101 and stores the system information in the storage device in the image processing device 101, for example, the condition storing unit 6 (step S0).

Next, steps S1 to S7 are executed in the same manner as in the first embodiment.

After the non-observed region detection in the three-dimensional model is temporarily stopped (step S7), the non-observed-region detection unit 14 may automatically resume the non-observed region detection. For example, the non-observed-region detection unit 14 may resume the non-observed region detection on the basis of a signal indicating an operation for tuning off the switch of the NBI, the RDI, or the EDOF.

After step S7, step S8 or step S10 and S11, described in the first embodiment, may be executed.

Accordingly, with the image processing device 101 according to this embodiment, the predetermined operations of the endoscope system 200 that generate a missing portion in the image group are detected by determining whether or not the operation signals of the endoscope system 200 match the certain conditions. After the predetermined operations are detected, the non-observed region detection is temporarily stopped. Accordingly, even in the case in which a missing portion is generated in the image group, the missing portion due to the image group missing a portion thereof is prevented from being erroneously detected as the non-observed region, and, accordingly, it is possible to prevent the occurrence of a problem in the non-observed region detection.

In addition, because the respective devices 20, 30, and 40 have different functions depending on the device models thereof, the operations of the endoscope system 200 that generate the missing portion in the image group are different for each of the combinations of the devices 20, 30, and 40. With this embodiment, it is possible to automatically set certain conditions that are suitable for the device model combinations of the devices 20, 30, and 40.

The modified examples described in the first embodiment may be applied to this embodiment.

Specifically, the certain conditions may additionally include the operations of the endoscope system in which the missing portion detection is unnecessary. In addition, the certain conditions may be set, from the plurality of conditions, on the basis of the user operations of the user interface 4 (see FIG. 7).

The display control unit 15 may create reports after the endoscopy is ended (see FIGS. 8A and 8B).

As above, the embodiments of the present disclosure and the modified examples thereof have been described in detail with reference to the drawings; however, the specific configurations of the present disclosure are not limited to the above-described embodiments and modified examples, and various design alterations are possible within the range that does not depart from the scope of the present disclosure. In addition, the constituent elements indicated in the above-described embodiments and modified examples can be combined, as appropriate.

For example, the imaging subject to be observed by means of the endoscope may be an organ other than the colon.

### Example 1

An image processing device that is used with an endoscope system and that processes images of an image subject captured by an endoscope, the image processing device comprising a processor, wherein
the processor is configured to:
   acquire operation signals of the endoscope system; and when the processor creates a three-dimensional model of the imaging subject from an image group of the images captured by the endoscope;
   select an image for generating the three-dimensional model of the imaging subject;
   detect a non-observed region in which an image thereof is not captured by the endoscope based on the three-dimensional model;
   wherein the certain condition includes an operation of the endoscope system that causes a portion in the image group to have a missing portion.

### Example 2

An image processing device that is used with an endoscope system and that processes images of an image subject captured by an endoscope, the image processing device comprising a processor, wherein
the processor is configured to:
create a three-dimensional model of the imaging subject from an image group of the images captured by the endoscope;
detect a non-observed region in which an image thereof is not captured by the endoscope based on the three-dimensional model;
acquire operation signals of the endoscope system; and
temporarily stops detecting the non-observed region in a case in which the operation signals match a certain condition, wherein the certain condition includes an operation of the endoscope system that causes a portion in the image group to have a missing portion.

### REFERENCE SIGNS LIST

1, 102 processor
2 storage unit (storage medium)
2a image processing program
4 user interface
6 condition storing unit (storage unit)
10, 101 image processing device
20 endoscope
22 zooming mechanism
23 angle sensor (sensor)
30 light source device
40 controller
61 air feeding pump
62 air suction pump
63 liquid feeding pump
64 liquid suction pump
65 high-frequency treatment tool
66 UPD (sensor)
C three-dimensional model
D non-observed region

What is claimed is:

1. An image processing device for use with an endoscope system, the image processing device comprising:
one or more processors comprising hardware, wherein the one or more processors being configured to:
create a three-dimensional model of a subject from a group of images captured by an endoscope in the endoscope system;
detect a non-observed region in which an image from the group of images is not captured by the endoscope based on the three-dimensional model;
acquire operation signals of the endoscope system; and
determine whether the operation signal matches a condition, wherein the condition includes an operation of the endoscope system that causes the group of images has a missing portion;
responsive to determining the operation signal matches the condition, temporarily stop detecting the non-observed region.

2. The image processing device according to claim 1, wherein:
the operation signals include a signal that indicates switching types of illumination light provided to the endoscope from a light source; and
the condition includes the switching types of the illumination light.

3. The image processing device according to claim 1, wherein the condition includes an operation of the endoscope system in which the non-observed region detection is unnecessary.

4. The image processing device according to claim 3, wherein:
the operation signals include a signal that indicates changing a magnification of a zooming mechanism of the endoscope; and
the condition includes changing the magnification of the endoscope.

5. The image processing device according to claim 3, wherein:
the operation signals include a signal that indicates an activation of one of an air feeding pump or an air suction pump; and
the condition includes the activation of the air feeding pump or the air suction pump.

6. The image processing device according to claim 3, wherein:
the operation signals include a signal that indicates an activation of one of a liquid feeding pump or a liquid suction pump; and
the condition includes the activation of one of the liquid feeding pump or the liquid suction pump.

7. The image processing device according to claim 3, wherein:
the operation signals include a signal that indicates an activation of a high-frequency treatment tool; and
the condition includes the activation of the high-frequency treatment tool.

8. The image processing device according to claim 3, wherein:
the operation signals include a signal from a sensor configured to detect a bending angle of a bending portion of the endoscope; and
the condition includes bending of the bending portion at a predetermined bending angle or more.

9. The image processing device according to claim 1 further comprising a user interface that accepts a user operation, wherein the one or more processors being configured to select at least one of a plurality of conditions based on the user operation accepted by the user interface and sets a selected condition to be the condition.

10. The image processing device according to claim 1 further comprising a storage, wherein:
the storage is configured to store a correspondence relationship between combinations of device models of a plurality of devices comprising the endoscope system and the condition, wherein the plurality of devices include the endoscope and a light source configured to provide illumination light to the endoscope; and
the one or more processors being configured to:
acquire device model information of each of the plurality of devices to which the image processing device is connected, and
acquire the condition corresponding to one of the combinations of the device models from the storage.

11. The image processing device according to claim 1, wherein subsequent to temporarily stopping detecting the non-observed region, the one or more processors being configured to remove a matching image that corresponds to the condition.

12. The image processing device according to claim 10, wherein subsequent to removing the matching image, the one or more processors being configured to detect the non-observed region.

13. An image processing method comprising:
creating a three-dimensional model of a subject from a group of images captured by an endoscope;
detecting a non-observed region in which an image from the group of images is not captured by the endoscope based on the three-dimensional model;
acquiring operation signals of an endoscope system including the endoscope;
determining whether the operation signal matches a condition, wherein the condition includes an operation of the endoscope system that causes the group of images has a missing portion; responsive to determining the operation signal matches the condition, temporarily stopping detection of the non-observed region.

14. The image processing method according to claim 12, wherein:
the operation signals include a signal that indicates switching types of illumination light provided to the endoscope from a light source; and
the condition includes the switching types of the illumination light.

15. The image processing method according to claim 12, wherein:
the condition includes an operation of the endoscope system in which the non-observed region detection is unnecessary.

16. The image processing method according to claim 12, wherein:
the operation signals include a signal that indicates changing a magnification of a zooming mechanism of the endoscope; and
the condition includes changing the magnification of the endoscope.

17. The image processing method according to claim 12, wherein:
the operation signals include a signal that indicates an activation of one of an air feeding pump or an air suction pump; and
the condition includes the activation of one of the air feeding pump or the air suction pump.

18. A computer-readable, non-transitory storage medium configured to cause a computer to execute:
creation of a three-dimensional model of a subject from a group of images captured by an endoscope;
detection of a non-observed region in which an image from the group of images is not captured by the endoscope based on the three-dimensional model;
acquisition of operation signals of an endoscope system including the endoscope; and
determination of whether the operation signal matches a condition, wherein the condition includes an operation of the endoscope system that causes the group of images has a missing portion;
responsive to determining the operation signal matches the condition, temporary stopping the detection of the non-observed region.

19. The computer-readable, non-transitory storage medium according to claim 17, wherein:
the operation signals include a signal that indicates switching types of illumination light provided to the endoscope from a light source; and
the condition includes the switching types of the illumination light.

20. The computer-readable, non-transitory storage medium according to claim 17, wherein:
the condition includes an operation of the endoscope system in which the non-observed region detection is unnecessary.
the condition includes changing the magnification of the endoscope.
